(19) United States
(12) Patent Application Publication
(10) Pub. No.: US 2010/0217009 A1
(43) Pub. Date: Aug. 26, 2010

(54) Process for Preparing Chroman Derivatives

(75) Inventors: David Alexander Learmonth, Alfena (PT); Alexander Beliaev, Mindelo (PT)

Correspondence Address: CONLEY ROSE, P.C., 5601 GRANITE PARKWAY, SUITE 750, PLANO, TX, 75024, US

(73) Assignee: BIAL - PORTELA & CA, S.A., S. Mamede do Coronado (PT)

(21) Appl. No.: 12/600,820

(22) PCT Filed: May 21, 2008
(86) PCT No.: PCT/PT08/00023

§ 371 Date: Mar. 11, 2010

(30) Foreign Application Priority Data
May 21, 2007 (GB) 0709695.1

(51) Int. Cl. C07D 405/14; C07D 311/58; C07C 229/22; C07C 271/22; C07D 311/22

(52) U.S. Cl. 548/312.1; 549/404; 562/444; 560/29; 549/401

(57) ABSTRACT

A process for preparing a compound of formula (22), comprising reducing a compound of formula (21), to produce a compound of formula (23), followed by the hydrogenolysis of the compound of formula 23 in a solvent comprising a C₁ to C₆ alkyl sulfonic acid and optionally a chlorinated solvent.

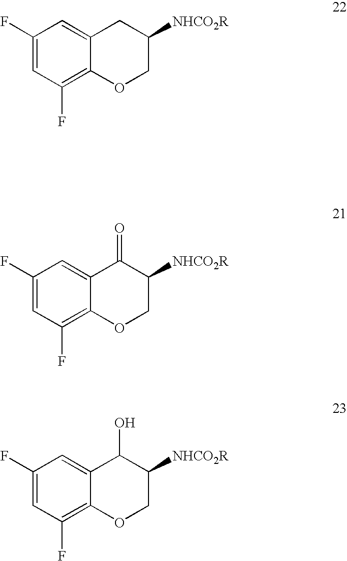

This invention relates to a method of making carbamic acid ethyl esters. Such carbamic acid esters are useful in the preparation of dopamine-β-hydroxylase inhibitors, as described in WO2004/033447.

The amine 8 depicted below is a useful intermediate in the preparation of the dopamine-β-hydroxylase inhibitors described in WO2004/033447. It may be synthesised starting from L-serine methyl ester hydrochloride (2) by condensation of its N-trityl derivative with 2,4-difluorophenol under Mitsunobu conditions (Cherney, R. L.; Wang, L. Efficient Mitsunobu reactions with N-phenylfluorenyl or N-trityl serine esters. J. Org. Chem. 1996, v. 61, N 7, p. 2544-2546) followed by deprotection, ethoxycarbonylation of the resulting amino acid (4), Friedel-Crafts cyclization (McClure, D. E.; Arison, B. H.; Jones, J. H.; Baldwin, J. J. Chiral α-amino ketones from the Friedel-Crafts reaction of protected amino acids. J. Org. Chem. 1981, v. 46, N 11, p. 2431-2433) of N-protected derivative (20) and reduction of the ethoxycarbonylamino ketone (21). The alkaline hydrolysis of ethyl carbamate (22) gives 8:

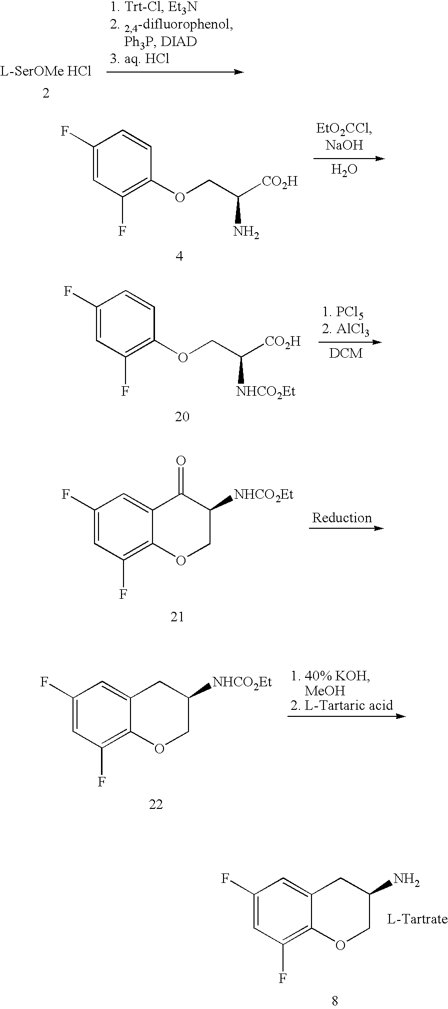

A similar reaction can be carried out with the other enantiomer, as follows:

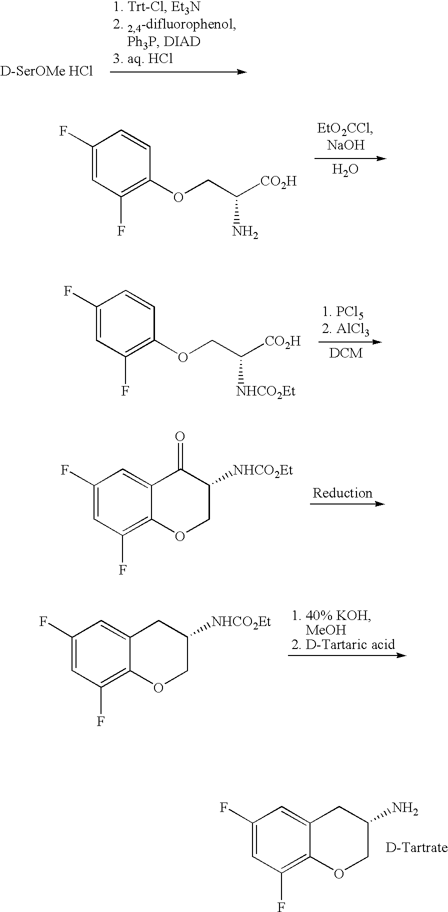

The second, third, fourth, fifth and sixth compounds in the scheme immediately above are compounds 4′, 20′, 21′, 22′ and 8′ respectively.

Reduction of aryl ketones (such as compound 21) to methylene compounds (such as compound 22) is a synthetically useful reaction and various methods have been developed for this transformation, namely Clemmensen and Wolff-Kishner reductions, catalytic hydrogenolysis (Sarda, N., Grouiller, A., Pacheco, H. Nouvelle synthese de l'amino-3-chromanne. Synthese et configuration absolue de ses enantiomeres. Tetrahedron Letters 1976, N 4, p. 271-272; Norlander, E. J., Njoroge, F. G., Payne, M. J., Warman, D. N-(Trifluoroacetyl)-α-amino acid chlorides as chiral reagents for Friedel-Crafts synthesis. J. Org. Chem. 1985, v. 50, N 19, p. 3481-3484; Norlander, E. J., Payne, M. J., Njoroge, F. G., Balk, M. A., Laikos, G. D., Vishvanath, V. M. Friedel-Crafts acylation with N-(trifluoroacetyl)-α-amino acid chlorides. Application to the preparation of β-arylalkylamines and 3-substituted 1,2,3,4-tetrahydroisoquinolines. J. Org. Chem. 1984, v. 49, N 32, p. 4107-4111), Et₃SiH/CF₃COOH [Norlander, E. J., Njoroge, F. G., Payne, M. J., Warman, D. N-(Trifluoroacetyl)-α-amino acid chlorides as chiral reagents for Friedel-Crafts synthesis. J. Org. Chem. 1985, v. 50, N 19, p. 3481-3484; Norlander, E. J., Payne, M. J., Njoroge, F. G., Balk, M. A., Laikos, G. D., Vishvanath, V. M. Friedel-Crafts acylation with N-(trifluoroacetyl)-α-amino acid chlorides. Application to the preparation of β-arylalkylamines and 3-substituted 1,2,3,4-tetrahydroisoquinolines. J. Org. Chem. 1984, v. 49, N 32, p. 4107-4111), Et₃SiH/BF₃.Et₂O (Norlander, E. J., Payne, M. J., Njoroge, F. G., Balk, M. A., Laikos, G. D., Vishvanath, V. M. Friedel-Crafts acylation with N-(trifluoroacetyl)-α-amino acid chlorides. Application to the preparation of (3-arylalkylamines and 3-substituted 1,2,3,4-tetrahydroisoquinolines. J. Org. Chem. 1984, v. 49, N 32, p. 4107-4111), Et₃SiH/TiCl₄ (Yato, M.; Homma, K.; Ishida, A. New silane reduction of aromatic ketones mediated by titanium tetrachloride: a synthesis of γ- and δ-aryl substituted amino acids. Heterocycles 1998, v. 49, p. 233-254), NaBH₄/BF₃Et₂O (Perry, P. J.; Pavlidis, V. H.; Coutts, I. G. C. The rapid reduction of α,α-diaryl alcohols to the corresponding alkanes using iodotrimethylsilane. Synth. Commun. 1996, v. 26, N 1, p. 101-111). None of the above methods proved to be efficient for the conversion of 21 to 22, yielding the intermediate alcohol or its mixture with the starting material. Attempts to reduce the intermediate alcohol to 22 with other reagents known to be efficient for this kind of transformation, like iodotrimethylsilane (Pettit, G. R.; Piatak, D. M. J. Org. Chem. 1962, v. 27, p. 2127) or its equivalents (Cain, G. A.; Holler, E. R. Extended scope of in situ iodotrimethysilane mediated selective reduction of benzylic alcohols. Chem. Commun. 2001, p. 1168-1169), did not work either. Improved results can be obtained using a type of scheme corresponding to the catalytic reduction method used previously for the preparation of the nepicastat intermediate (U.S. Pat. No. 5,438,150):

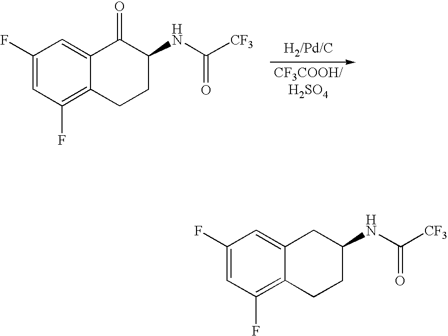

However, the yield of compound 22 does not exceed 50%, the major by-product being the corresponding trifluoroacetylated alcohol:

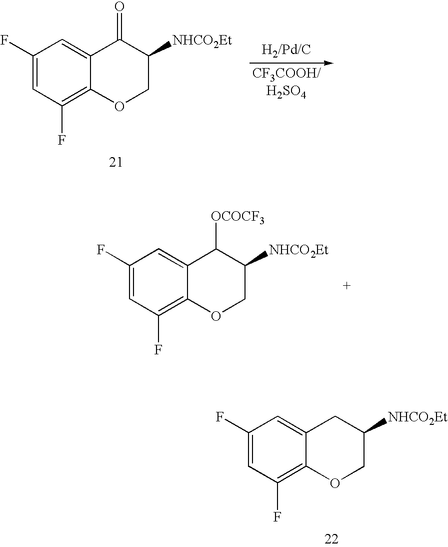

Besides, using the highly corrosive mixture of trifluoroacetic and sulphuric acids on larger scale could be problematic. An attempt to replace trifluoroacetic acid with acetic acid results in low yield of 22 with significant quantities of the acetoxy by-product.

We have surprisingly found that carrying out the reduction in C₁ to C₆ alkyl sulfonic acid, such as methanesulfonic acid, or preferably in a mixture of 3 volumes of a chlorinated solvent, such as dichloromethane, and 1 volume of a C₁ to C₆ alkyl sulfonic acid, such as methanesulfonic acid, gives complete conversion of 21 to 22.

However, the analysis of the optical purity of compound 22 revealed significant racemisation. We have found that the cause of the racemisation is the enantiomeric instability of the starting material in the mixture of 3 volumes of a chlorinated solvent, such as dichloromethane, and 1 volume of methanesulfonic acid. Based on these results, for situations where optical purity is important, a two-step method was devised comprising converting 21 to the intermediate alcohol, with sodium borohydride followed by catalytic reduction in the mixture of 3 volumes of dichloromethane and 1 volume of methanesulfonic acid. The two-step process reproducibly gave high yield of compound 22 with 94-99% ee.

Thus, according to one aspect of the invention there is provided a process for preparing a compound of formula 22:

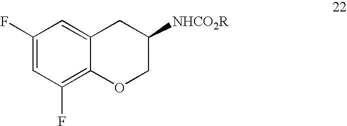

comprising reducing a compound of formula 21:

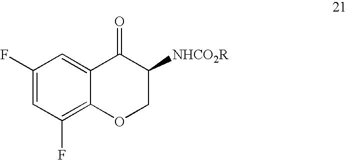

to produce a compound of formula 23:

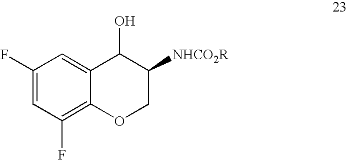

followed by the hydrogenolysis of the compound of formula 23 in a solvent comprising a C₁ to C₆ alkyl sulfonic acid, wherein R is alkyl or aryl, wherein: the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; and the term halogen means fluorine, chlorine, bromine or iodine.

Preferably R is ethyl, methyl, tBu or benzyl, most preferably ethyl.

Preferably the alkyl sulphonic acid is methanesulfonic acid.

Although it is possible to carry out the reaction in a solvent of substantially pure alkylsulfonic acid, it is preferred that the reaction is carried out in a solvent mixture comprising a chlorinated solvent and a C₁ to C₆ alkyl sulfonic acid. Again, it is preferred that the alkylsulfonic acid is methansulfonic acid. We prefer that the ratio of chlorinated solvent to alkylsulfonic acid solvent is not more than 10:1, more preferably not more than 5:1, still more preferably in the range 4:1 to 2:1. Most preferably the ratio of chlorinated solvent to alkylsulfonic acid solvent is about 3:1.

In general terms, the first step of the process comprises reducing (R)-(6,8-difluoro-4-oxochroman-3-yl)carbamic acid ethyl ester (21). This may be done using a borohydride, particularly an alkali metal borohydride, such as sodium borohydride, in a suitable solvent. The solvent may be a lower alcohol, optionally in combination with THF. The lower alcohol may be a linear or branched alcohol having from 1 to 6 carbon atoms; the lower alcohol may be ethanol, or, most preferably, methanol. This reduction may suitably be carried out at 5-25° C. for 0.5-4 hours.

The reduction step is followed by hydrogenolysis of the intermediate alcohol, preferably over a palladium catalyst (7% w/w of the water free 10% Pd on activated carbon may be used). The reaction may suitably be carried out at 15-100 psi of hydrogen pressure and at a temperature of 20-25° C. in the solvent mixture for 7 to 26 hours. We have found that using less than 1 volume of methanesulfonic acid leads to lower conversion.

When the ratio of chlorinated solvent to alkylsulfonic acid solvent is less than 5:1 the reaction may require 10% w/w of the Pd with a hydrogen pressure of 100 psi and a reaction time of 15 hours. Thus, a higher amount of the chlorinated solvent tends to increase the reaction time, the hydrogen pressure and the amount of Pd needed.

The chlorinated solvent is advantageously an alkyl halide, preferably an C₁-C₆ alkyl halide, more preferably a methyl or ethyl halide. The halide is preferably a chloride. The chlorinated solvent is most preferably dichloromethane or a dichloromethane-chloroform mixture. Alternatively, the chlorinated solvent may be 1,2-dichloroethane.

According to another aspect of the invention there is provided a process, for preparing a compound of formula 8, comprising forming a compound of formula 22 using a process as described above, followed by alkaline hydrolysis of the compound of formula 22.

The alkaline hydrolysis step may be carried out in the presence of an alkaline earth metal hydroxide or an alkali metal hydroxide; an alcohol having 1 to 6 carbon atoms; and L-tartaric acid. The alkali metal hydroxide is preferably potassium hydroxide. The alcohol is preferably methanol.

According to another aspect of the invention there is provided a process for preparing a compound of formula 14:

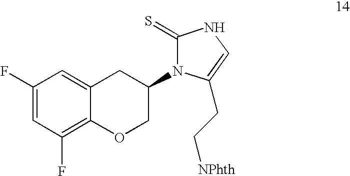

comprising preparing a compound of formula 8, for example by a process described above, then reacting the compound of formula 8 with a compound of formula 13:

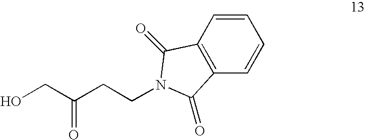

Preferably, the reaction is carried out in the presence of an alkali metal isothiocyanate, preferably potassium isothiocyanate, and an organic acid, preferably acetic acid.

According to another aspect of the invention there is provided a process for preparing a compound of formula 1:

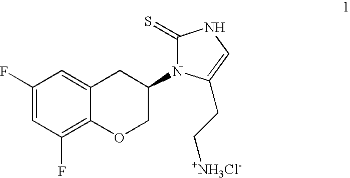

comprising preparing a compound of formula 14 by a process as described above, then converting the compound of formula 14 to the compound of formula 1 with an alkali metal borohydride, preferably sodium borohydride (NaBH₄), in the presence of a solvent, followed by adding HCl then recovering the compound of formula 1.

Preferably, the solvent used in the conversion of compound 14 is a mixture of isopropyl alcohol, water and dichloromethane.

According to further aspects of the invention, there is provided compounds of formulas, 4, 4′, 8, 8′, 20, 20′, 21, 21′, 23 and 23′. In an embodiment, said compounds are provided in substantially isolated form.

The invention will be further described with reference to the following examples.

### EXAMPLES

### Example 1

### (R)-2-Amino-3-(2,4-difluorophenoxy)propionic acid (4)

The vessel was purged with nitrogen followed by a charge of L-serine methyl ester hydrochloride (25 kg) and dichloromethane (400 kg, 300 L). The temperature of the vessel contents were maintained using glycol cooling (at a temperature ranging from 15-25° C.). Triethylamine (33.4 kg) was charged to the vessel over 45 min. A solution of trityl chloride (45.7 kg) in dichloromethane (265 kg) was prepared and charged to the vessel over 3 hours maintaining the temperature between 15-25° C. The resultant reaction mixture was stirred for 6 hours at 25-30° C. HPLC analysis confirmed complete reaction.

Water (263 kg) was charged to the vessel and the mixture stirred for 30 minutes and allowed to settle 30 minutes. The lower organic phase was separated off and then the top aqueous phase was extracted with DCM (90 kg). The combined organic phase was recharged to the vessel (after removal of aqueous phase) and toluene (450 kg) was added. The DCM was distilled out using vacuum distillation (base temperature less than 35° C. and atmospheric pressure used initially, followed by application of vacuum down to 200 mbar). The distillate was monitored for DCM/toluene content by gas chromatography.

The contents of the vessel were then cooled to below 30° C. using cooling water and the reactor was vented with nitrogen. 2,4-Difluorophenol (21.3 kg) was charged to the vessel followed by triphenylphoshine (42.4 kg). The mixture was stirred out for 30 min. Maintaining the reaction temperature in the range 25-30° C., the diisopropyl azodicarboxylate (DIAD, 40.9 kg) was charged over 3 hours and 30 min. The reaction mixture was stirred for a further 4 hours before sampling. The reaction was analysed by HPLC and showed no starting material.

6N Hydrochloric acid (400 kg) was charged to the reaction mixture in the vessel and the mixture was warmed gently to reflux (observed temp=79.3° C.). The mixture was held at reflux for a further 4 hours before being cooled to 60-65° C. The mixture was allowed to settle for 1 hour at 60-65° C. before separating off the lower aqueous phase. The organic phase was extracted with 2N hydrochloric acid (20 kg) at 60-65° C. and the aqueous phase was combined with the first, lower aqueous phase.

The combined aqueous phase was cooled to 20-30° C. and then the pH was adjusted to pH 6.8-7.2 using 32% w/w sodium hydroxide solution (294.5 kg used). The resultant suspension was stirred for 1 hour and the pH was checked and adjusted to 6.8-7.2 as necessary. The solids were filtered off and the filter cake was washed with water (175 L). The solid on the filter was then re-slurried with acetone (140 kg) and filtered. The solid was pulled down as dry as possible (21.3 kg damp weight) and then dried at 40-45° C./100-60 mbar.

Yield=15.25 kg (as dry product)

### Example 2

### (R)-(6,8-Difluoro-4-oxochroman-3-yl)carbamic acid ethyl ester (21)

The vessel was purged with nitrogen and charged with 1M sodium hydroxide (120 kg) followed by compound 4 (15.25 kg). Ethyl chloroformate (9.5 kg) was charged to the vessel and the mixture was cooled to 0-10° C. The pH of the mixture was adjusted to 8.9-9.1 using 1N sodium hydroxide (35 kg). The resultant mixture was stirred for 2 hours at 0-10° C. The mixture was sampled and analysed by thin layer chromatography (TLC). The reaction was incomplete and a further 14.5 kg of 1N sodium hydroxide was added. That the reaction was complete was confirmed by TLC.

Dichloromethane (265 kg) was charged to the vessel. The pH was adjusted to 0.9-1.2 using 6M hydrochloric acid (20 kg) with cooling. The resultant mixture was stirred for a further hour. The pH was checked and did not need further adjustment. After settling for 1 hour the lower organic phase was dropped out. After emptying the vessel the organic phase was washed with 2M hydrochloric acid (60 kg), and saturated brine (60 kg).

The organic phase was then charged to vessel and the volume was reduced by approximately 100 L using vacuum distillation. Temperature was not allowed to rise above 35° C. (atmospheric pressure initially used followed by vacuum to 380 mbar). The aim of the strip step was to dry the organic phase. Solvent strip took 5 hours. Karl Fisher analysis of the mixture was 0.1445% water.

The mixture was then cooled and removed to drums. The vessel was then charged with phosphorus pentachloride (16 kg) and dichloromethane (265 kg). The suspension was cooled to 0-2° C. Then the solution of protected amine was added over 3 hours maintaining a reaction temperature in the range of 0-5° C. The resultant reaction mixture was stirred for further 4 hours in the temperature range of 0-5° C. The crude DCM solution intermediate was discharged to clean dry drums.

Vessel was charged with dichloromethane (265 kg) and aluminium chloride (25.1 kg) and the suspension was cooled to 3-5° C. The drummed solution was charged to the vessel, maintaining a temperature range of 3-8° C. The addition took 1.5 hours and the mixture was then stirred for further 6 hours. Meanwhile hydrochloric acid solution at approx 10% w/w (water 285 kg and conc HCl 31 kg) was prepared in a second vessel and chilled to 0° C. The solution in the first vessel was quenched into the cold acid solution maintaining temperature below 10° C. There was no substantial solid aluminium chloride remaining; only a few grains of solid were present. The quench took 3.25 hours followed by an hour stir out and an hour to settle. The organic phase was separated off and the remaining aqueous phase was extracted with dichloromethane (60 kg). The organic phases were combined and washed with 2M hydrochloric acid (230 kg), water (230 kg), 10% bicarbonate solution (230 kg), followed by saturated brine (95 kg). The organic phase was recharged to vessel for solvent concentration and then was transferred to 25 litre containers for evaporation in a 20 litre rotary evaporator. The product was evaporated to a damp solid to enable transfer to trays so that the product could be dried in a vacuum oven at 30° C. to remove the remaining traces of solvent.

Yield=13.2 kg, enantiomeric excess 99.6% by chiral HPLC. The following conditions were used for the chiral HPLC: column ChiralPak AD-H, wavelength 210 nm, injection volume 4 μl, flow: 0.3 ml/min, eluent: 30:70 MeOH:IPA.

### Example 3

### (R)-(6,8-Difluorochroman-3-yl)carbamic acid ethyl ester (22)

To a suspension of compound 21 (36 g, 132.7 mmol) in methanol (265 ml) sodium borohydride (5.5 g, 144.7 mmol) was added portion-wise during 20 min, maintaining the reaction temperature in the range of 5-10° C. The mixture was allowed to warm up to 20° C. during 30 min, water (20 ml) was added portion-wise and the mixture was evaporated to dryness under reduced pressure. The residue was distributed between ethyl acetate (250 ml) and 10% brine (250 ml). The organic phase was dried (MgSO₄) and evaporated to dryness under reduced pressure. The resulting oil (37.5 g) was hydrogenated in the mixture of dichloromethane (260 ml) and methanesulfonic acid (87 ml) over 10% Pd/C (2.6 g) at 100 psi of hydrogen pressure and 20-25° C. for 7 h. The catalyst was filtered on Celite™, the Celite™ pad was washed with dichloromethane (65 ml), water (65 ml) and dichloromethane (65 ml). 4N NaOH (348 ml) was added to the filtrate with stirring and ice-cooling maintaining the temperature below 30° C. The organic phase was washed with brine (100 ml), dried (MgSO₄) and evaporated to dryness. Yield 28 g (82%), enantiomeric excess 98% by chiral HPLC. AB: The following conditions were used for the chiral HPLC: column ChiralPak AD-H, wavelength 210 nm, injection volume 20 μl, flow: 0.5 ml/min, eluent: 70:30 MeOHIPA.

### Example 4

### (R)-(6,8-Difluorochroman-3-yl)carbamic acid ethyl ester (22)

The vessel was purged with nitrogen. Compound 21 (13.2 kg) was charged to the vessel followed by methanol (155 kg). The suspension was cooled to 5-10° C. then sodium borohydride (2.1 kg) was added portion wise (200 g) to the vessel maintaining the reaction temperature in the range of 5-10° C. Gas evolution was observed and it was ensured that the effervescence had stopped before the next portion of sodium borohydride was added. Addition of sodium borohydride took 4 hours. The reaction mixture was stirred for 4 hours maintaining the temperature in the range 5-10° C. The sample taken showed that no starting material remained.

Acetone (20 kg) was charged as a quench and the mixture was stirred for a further 2 hours. The methanol/acetone volume was reduced using vacuum distillation (base temp. not greater than 45° C. and atmospheric pressure used initially followed by application of vacuum down to 170 mbar). The strip took 6.5 hours and the concentrated solution was then transferred to 25 litre containers for further evaporation in the laboratories. A thick, sticky residue was obtained with weight of 21.9 kg. The residue was dissolved in dichloromethane (300 kg) and the organic mixture was extracted with 10% brine solution (100 kg). The brine solution was then back extracted with dichloromethane (100 kg). The combined organic phase was charged to vessel. The vessel was twice purged with nitrogen to 0.5 bar. The catalyst 10% palladium on charcoal (1.25 kg on dry basis) was charged to the vessel followed by methanesulfonic acid (72 kg). The vessel was pressured to 0.5 bar with nitrogen with a final pressure check. Then the vessel was pressured to 0.5 bar three times with hydrogen. The agitator was started and the pressure increased to 1.2 bar. The mixture was allowed to hydrogenate for 26 hours. After purging with nitrogen the mixture was filtered with the aid of Celite™. The organic phase was kept separate. The Celite™ and catalyst were then washed with water (100 kg). The water was recharged to the cleaned vessel and the dichloromethane phase was added very slowly to the vessel with cooling. Large exotherms were observed in the mixing of the organic and aqueous phases. Once the two phases were mixed, the mixture was cooled to 5-10° C. before the pH was adjusted with 4N sodium hydroxide (approx 172 kg added) to a pH of 10. Cooling was required and the pH adjustment took 2.5 hours. The mixture was filtered with the aid of Celite™ and rinsed with water (30 kg). The mixture was charged to a vessel to allow settling and then the organic phase was removed. The volume of the organic phase was reduced to 60 L using vacuum distillation taking 1.5 hours (base temp. not greater than 40° C. and atmospheric pressure used initially followed by application of vacuum down to 200 mbar). The concentrate was then transferred to the 20 litre rotary evaporator for product isolation. The product was put into oven under vacuum at 30° C. for drying off any traces of solvent. Yield 7.87 kg, enantiomeric excess 94.1% by chiral HPLC. The following conditions were used for the chiral HPLC: column ChiralPak AD-H, wavelength 210 nm, injection volume 20 μl, flow: 0.5 ml/min, eluent: 70:30 MeOH:IPA.

### Example 5

### (R)-(6,8-Difluorochroman-3-yl)carbamic acid ethyl ester (22)

Compound 21 (30 g) was taken into methanol (250 ml), and sodium borohydride (4.8 g) was added portion-wise, maintaining the reaction temperature in the range of 5-10° C. A solution was observed after all the sodium borohydride had been added. The mixture was allowed to stir at room temperature for 2 hours. Acetone (20 ml) was added to the reaction mixture to quench it and the mixture was stirred overnight. Methanol was distilled off at atmospheric pressure with periodic addition of chloroform until methanol content in the distillate reached 0.7% (GC), the total volume was adjusted with chloroform to 250 ml. The mixture was washed with 10% brine (200 ml), the organic phase was analysed (Karl Fisher=0.20% water), mixed with dichloromethane (100 ml) and methanesulfonic acid (117 g), and hydrogenated over 10% Pd/C (2.93 g) at 5 bar and 25° C. for 16 h. The reaction was not complete by HPLC therefore a further 53 g of methanesulfonic acid was added and the hydrogenation was continued for another 8 h. The catalyst was filtered on Celite™ and the Celite™ pad washed with dichloromethane (65 ml), water (60 ml) and dichloromethane (60 ml). 4N NaOH (340 ml) was added to the filtrate with stirring, and ice-cooling maintaining the temperature below 30° C. The organic phase was washed with brine (100 ml), dried (MgSO₄) and evaporated to dryness. Yield 23.5 g (83%), enantiomeric excess 97% by chiral HPLC. The following conditions were used for the chiral HPLC: column ChiralPak AD-H, wavelength 210 nm, injection volume 20 μl, flow: 0.5 ml/min, eluent: 70:30 MeOH:IPA.

### Example 6

### (R)-(6,8-Difluorochroman-3-yl)carbamic acid ethyl ester (22)

Sodium borohydride (0.04 g, 1 mmol) was added to a suspension of compound 21 (0.14 g, 0.5 mmol) in the mixture of methanol (0.5 ml) and THF (0.5 ml) whilst maintaining the reaction temperature in the range of 5-10° C. The mixture was allowed to warm up to 20° C. for 30 min. Water (1 ml) was added portion-wise and the mixture was evaporated to dryness under reduced pressure. The residue was distributed between ethyl acetate (2 ml) and 10% brine (2 ml), the organic phase was dried (MgSO₄) and evaporated to dryness under reduced pressure. The resulting oil (0.13 g) was hydrogenated in the mixture of dichloromethane (1 ml) and methanesulfonic acid (0.33 ml) over 10% Pd/C (0.01 g) at 100 psi of hydrogen pressure and 20-25° C. for 16 h. The catalyst was filtered on Celite™ and the Celite™ pad was washed with dichloromethane (2 ml), water (2 ml) and dichloromethane (2 ml). 4N NaOH (1.5 ml) was added to the filtrate with stirring, and ice-cooling maintaining the temperature below 30° C. The organic phase was washed with brine (2 ml), dried (MgSO₄) and evaporated to dryness. Yield 0.087 g (69%), enantiomeric excess 96% by chiral HPLC. AB: The following conditions were used for the chiral HPLC: column ChiralPak AD-H, wavelength 210 nm, injection volume 20 μl, flow: 0.5 ml/min, eluent: 70:30 MeOH:IPA.

### Example 7

### (R)-6,8-difluorochroman-3-ylamine L-tartrate (8)

The vessel was purged with nitrogen and then compound 22 (13.7 kg) was charged followed by methanol (86 kg). The mixture was warmed to 63-65° C. Potassium hydroxide (40%, 51 kg) solution was charged in one portion and after 15 minutes a second portion of 40% potassium hydroxide (44 kg) was added over a period of 30 minutes whilst maintaining the reaction temperature in the range of 63-65° C. The resultant mixture was stirred for a further 24 hours whilst maintaining the reaction temperature in the range of 65-70° C. The mixture was then cooled and water was charged (44 kg). The mixture was re-heated and methanol was removed using vacuum distillation (base temperature not greater than 70° C. and atmospheric pressure used initially followed by application of vacuum down to 125 mbar). The distillation took 5.5 hours. The reaction mixture was cooled to 30° C. and the remaining aqueous phase was extracted with toluene (152 kg) twice. The combined organic phase was reduced in volume to approx. 60 L by vacuum distillation (3.5 hours). Denatured ethanol was charged (395 kg) and the distillation was continued until the toluene level was below 2% (determined using a standard GC analysis). The vessel contents were cooled to 20° C. and the volume was adjusted with fresh denatured ethanol to the original volume of denatured ethanol charged. A solution of L-tartaric acid (8.86 kg) in water (72 kg) was prepared and this solution was charged over a period of 1 hour maintaining the temperature between 15-25° C. The resultant precipitate was stirred for 1 hour. The vessel contents were then filtered and the cake was washed with denatured ethanol (200 L). The product was dried at 45-50° C. at 60-100 mbar vacuum. Yield=15.1 kg, enantiomeric excess 99.0% by chiral HPLC. The following conditions were used for the chiral HPLC: column ChiralPak AD-H, wavelength 210 nm, injection volume 20 μl, flow: 0.5 ml/min, eluent: 70:30 MeOH:IPA with 0.2% tert-butylamine.

### Example 8

### Preparation of (R)-2-{2-[3-(6,8-Difluorochroman-3-yl)-2-thioxo-2,3-dihydro-1H-imidazol-4-yl]ethylisoindole-1,3-dione (14)

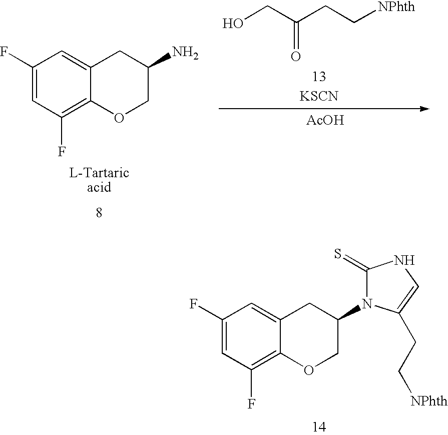

| | | |
|---|---|---|
| Reagents and solvents: | Aminochroman tartrate 8 | 4.02 g (12.0 mmol) |
| | Hydroxy ketone 13 | 3.36 g (14.4 mmol) |
| | Potassium thiocyanate | 1.40 g (14.4 mmol) |
| | Acetic acid | 48 mL |

A reactor was charged with solid reagents, acetic acid was added in one portion, and the mixture was heated to 105-110° C. under nitrogen with stirring and maintained under these conditions for 2 h. Water (24 mL) was added slowly with heating at ca. 90° C. (crystallisation occurred). The suspension was cooled in the ice-bath with stirring, stirred for 0.5 h in ice, water (24 mL) was added slowly and stirring continued for 1 h. The precipitate was collected, washed with AcOH-water (1:1 v/v), and water, then dried at 50-60° C. in vacuum. The resulting solid (5.25 g, 99%) was dissolved under reflux in the mixture of IPA (48 mL) and DCM (72 mL). The insoluble material (K tartrate) was filtered off, and the filtrate evaporated on a rotavap at ca. 50° C. and 500 mbar until crystallisation occurred. The heating was then removed and evaporation was continued to remove the residual DCM, using a vacuum meter to monitor the residual pressure. The suspension was left in the fridge overnight, the crystals collected and washed with IPA before drying in vacuum at 50° C. Yield 3.40 g (64%).

### Example 9

### Preparation of (R)-5-(2-aminoethyl)-1-(6,8-difluorochroman-3-yl)-1,3-dihydroimidazole-2-thione hydrochloride (BIA 5-453) (1), modification for Form A preparation

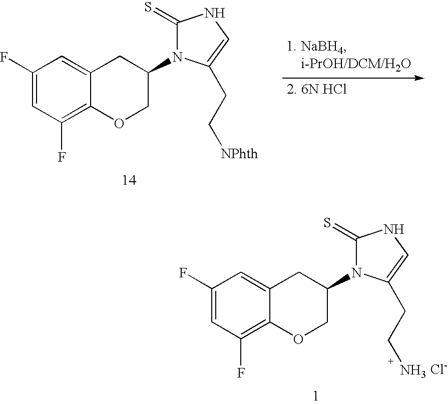

| | | |
|---|---|---|
| Reagents and solvents: | Phthalimide derivative 14 | 19.8 g (44.85 mmol) |
| | Sodium borohydride | 8.50 g ((224.25 mmo1) |
| | 2-Propanol | 396 mL |
| | Water | 69 mL |
| | Dichloromethane | 50 mL |

NaBH₄ was added in portions to a suspension of 14 in a′ mixture of 2-propanol, water and DCM at 18° C. during 1 min with stirring (temp. rose to 27.5° C. after 1 h). The mixture was stirred at 18-20° C. for 16 h (almost clear solution in 1 h). The mixture was cooled in the ice bath and 6N HCl (39.6 mL, 237.6 mmol) was added dropwise, keeping the temp. below 10° C. The mixture was stirred for 15 min, the solid filtered off, and the filter cake washed with DCM (300 mL) (4.9 g of solid were obtained). 5N NaOH (60 mL) was added to the mother liquor and the mixture was stirred for 15 min. The organic upper layer was separated, washed with brine, and filtered to remove minimum amount of solid. 6N HCl (40 ml) was added to the resulting clear solution, DCM was distilled off until the vapour temperature reached 76-78° C. and the mixture was stirred under reflux for 1.5 h and cooled to room temperature. Water (300 mL) was added, and the IPA was removed on a rotavap (420 mL were collected). The residue was then washed with EtOAc-petroleum ether (2:1 v/v) mixture (200 and 100 mL). After second washing, crystallisation started in aqueous phase. 6N HCl (40 mL) was added and the suspension was cooled in ice for 1 h with stirring. The precipitate was collected, washed with cold 3N HCl (75 mL) and cold IPA (50 mL), then dried in vacuum at 50° C. Yield 11.58 g (73%).

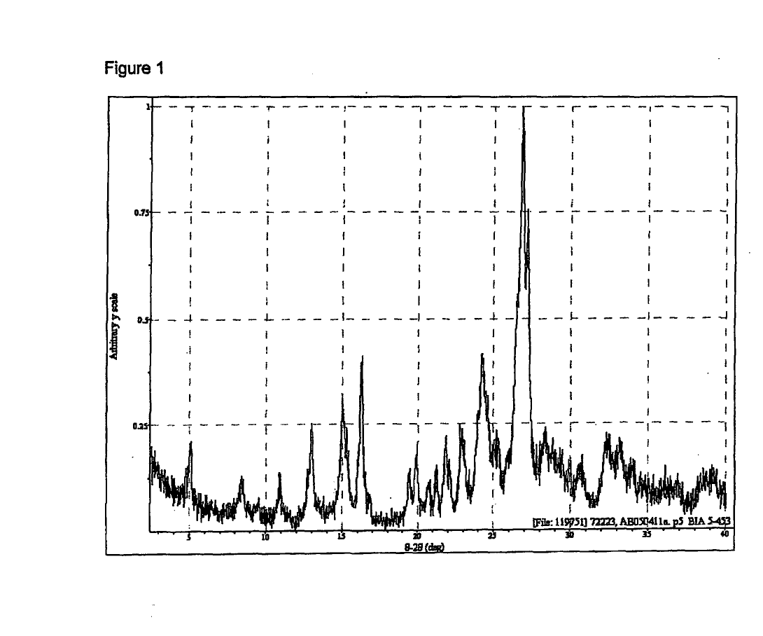

The crystalline form A of (R)-5-(2-Aminoethyl)-1-(6,8-difluorochroman-3-yl)-1,3-dihydroimidazole-2-thione hydrochloride has an XRPD pattern with peaks at 4.9, 8.3, 12.9, 15.0, 16.2, 19.8, 21.8, 22.9 and 24.2 26.8±0.2°2θ. The XRPD pattern is shown in FIG. 1.

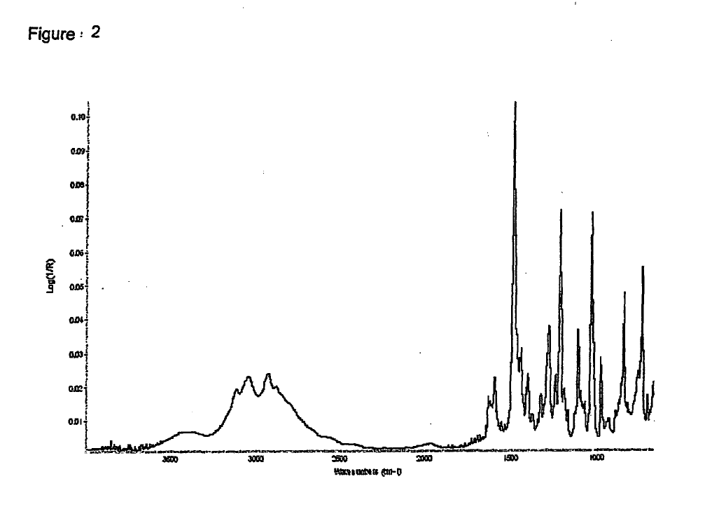

Furthermore, crystalline form A of (R)-5-(2-Aminoethyl)-1-(6,8-difluorochroman-3-yl)-1,3-dihydroimidazole-2-thione hydrochloride has characteristic FT-IR peaks at 3053.30, 2939.70, 1599.80, 1491.90, 1448.30, 1406.10, 1330.70, 1287.60, 1244.50, 1220.70, 1194.00, 1117.50, 1039.50, 985.50, 851.80, 747.00 and 713.70 cm⁻¹. The FT-IR spectrum is shown in FIG. 2.

### Example 10

### Preparation of (R)-5-(2-aminoethyl)-1-(6,8-difluorochroman-3-yl)-1,3-dihydroimidazole-2-thione hydrochloride (BIA 5-453) (1), Modification for Form. C Preparation

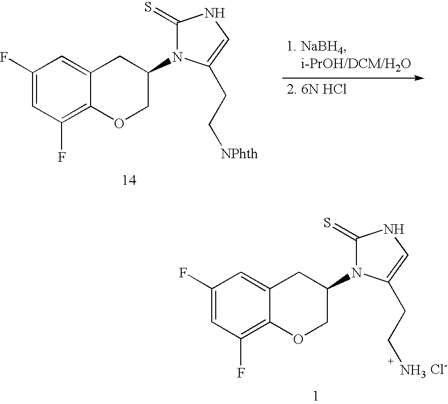

| | | |
|---|---|---|
| Reagents and solvents: | Phthalimide derivative 14 | 2.0 g (4.53 mmol) |
| | Sodium borohydride | 0.86 g ((22.64 mmol) |
| | 2-Propanol | 40 mL |
| | Water | 7 mL |
| | Dichloromethane | 5 mL |
| | 6N HCl | 8 mL |

NaBH₄ was added in portions to a suspension of 14 in the mixture of 2-propanol, water and DCM at 20° C. during 1 min with stirring (temp. rose to 22° C.). The mixture was stirred at 20° C. for 16 h (clear solution was produced after 0.5 h), and 6N HCl was added dropwise. DCM was distilled off until head temperature reached 76-78° C., and the mixture was stirred under reflux for 1.5 h and cooled to room temperature. Water (30 mL) was added. 2-propanol was removed on a rotavap, and the residue was twice washed with EtOAc-petroleum ether (2:1 v/v) mixture (2×20 mL). 10% 2-propanol in DCM solution (40 mL) was added to the aqueous layer with stirring, followed by addition of 5N NaOH to give pH 9-10. The organic layer was separated, dried (MgSO₄), evaporated to dryness. The residue was dissolved with heating in a mixture of abs. EtOH (15 mL) and 3M HCl in abs. EtOH (1.5 mL, pH of the mixture ca. 2). The resulting solution was stirred at 65-70° C. for 2 h, the crystals were collected, washed with EtOH, dried in vacuum at 40° C. Yield 1.12 g (71%).

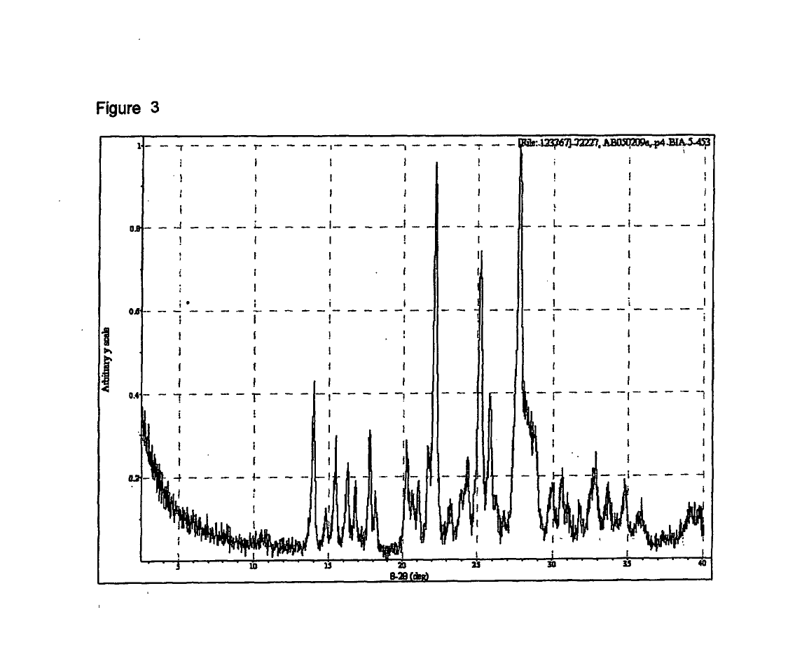

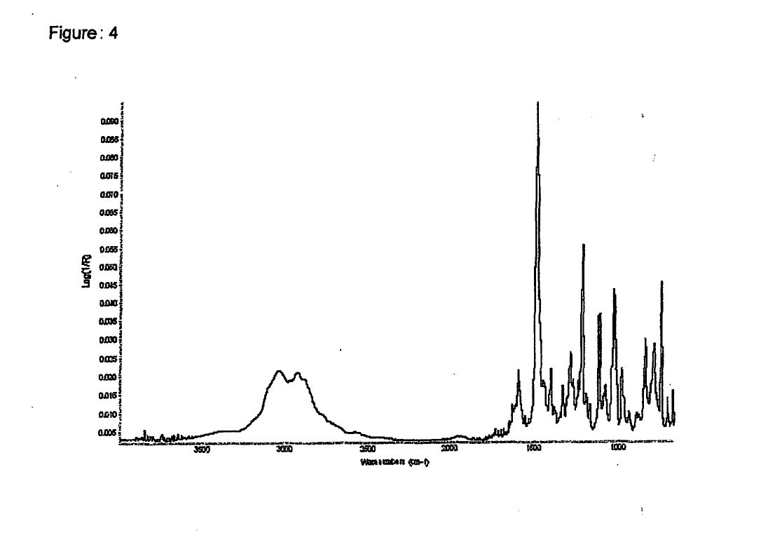

The crystalline form C of (R)-5-(2-Aminoethyl)-1-(6,8-difluorochroman-3-yl)-1,3-dihydroimidazole-2-thione hydrochloride has an XRPD pattern with peaks at 13.9, 15.3, 16.2, 16.7, 17.7, 18.1, 20.2, 21.0, 22.1, 24.2, 25.1 and 25.7. The XRPD pattern is shown in FIG. 3. Furthermore, the crystalline form C of (R)-5-(2-Aminoethyl)-1-(6,8-difluorochroman-3-yl)-1,3-dihydroimidazole-2-thione hydrochloride has characteristic FT-IR peaks at 3041.70, 1596.50, 1492, 1403.40, 1333.80, 1290.90, 1220.2, 1173.20, 1117.4, 1078.10, 1033.4, 984.90, 845.2, 792.6, 750.1 and 713.20 cm⁻¹. The FT-IR spectrum is shown in FIG. 4.

X-Ray Powder Diffraction (XRPD)

The following Shimadzu parameters were used to generate XRPD peak lists for Forms A and C.

| | | |
|---|---|---|
| Measurement Condition | Form A | Form C |

X-ray tube

| target | Cu | | Cu | |
|---|---|---|---|---|
| voltage | 40.0 | (kV) | 40.0 | (kV) |
| current | 40.0 | (mA) | 40.0 | (mA) |

Slits

| | | | | |
|---|---|---|---|---|
| divergence slit | 1.00000 | (deg) | 1.00000 | (deg) |
| scatter slit | 1.00000 | (deg) | 1.00000 | (deg) |
| receiving slit | 0.15000 | (mm) | 0.15000 | (mm) |

Scanning

| drive axis | 2Theta/Theta | | 2Theta/Theta | |
|---|---|---|---|---|
| scan range | 2.500-40.000 | | 2.500-40.000 | |
| scan mode | Continuous Scan | | Continuous Scan | |
| scan speed | 3.0000 | (deg/min) | 3.0000 | (deg/min) |
| sampling pitch | 0.0200 | (deg) | 0.0200 | (deg) |
| preset time | 0.40 | (sec) | 0.40 | (sec) |

Data Process Condition

| Smoothing | [MANUAL] | | [AUTO] | |
|---|---|---|---|---|
| smoothing points | 35 | | 19 | |
| B.G. Subtraction | [AUTO] | | [AUTO] | |
| sampling points | 45 | | 21 | |
| repeat times | 30 | | 30 | |
| Ka1-a2 Separate | [MANUAL] | | | [MANUAL] |
| Ka1 a2 ratio | 50.0 | (%) | 50.0 | (%) |
| Peak Search | [AUTO] | | [AUTO] | |
| differential points | 39 | | 19 | |
| FWHM threshold | 0.050 | (deg) | 0.050 | (deg) |
| intensity threshold | 30 | (par mil) | 30 | (par mil) |

| | | |
|---|---|---|
| FWHM ratio (n − 1)/n | 2 | 2 |
| System Error Correction | [NO] | [NO] |
| Precise Peak Correction | [NO] | [NO] |

FT-IR Spectroscopy

Infrared spectra were acquired on a Magna-IR 860® Fourier transform infrared (FT-IR) spectrophotometer (Thermo Nicolet) equipped with an Ever-Glo mid/far IR source, an extended range potassium bromide (KBr) beamsplitter, and a deuterated triglycine sulfate (DTGS) detector. A Thunderdome accessory was used for sampling. A background data set was acquired with a clean Ge crystal. A Log 1/R(R=reflectance) spectrum was acquired by taking a ratio of these two data sets against each other. Wavelength calibration was performed using polystyrene. Further parameters were as follows.

| | Form A | Form C |
|---|---|---|
| Number of sample scans | 128 | 256 |
| Number of background scans | 128 | 256 |
| Resolution/cm⁻¹ | 4.000 | 4.000 |
| Sample gain | 2.0 | 2.0 |
| Mirror velocity | 0.6329 | 0.6329 |
| Aperture | 100.00 | 100.00 |
| Detector | DTGS KBr | DTGS KBr |
| Beamsplitter | XT-KBr | XT-KBr |

1. A process for preparing a compound of formula 22: 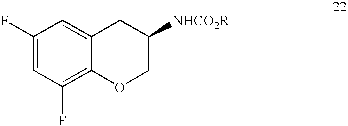
comprising reducing a compound of formula 21: 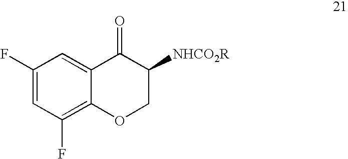
to produce a compound of formula 23: 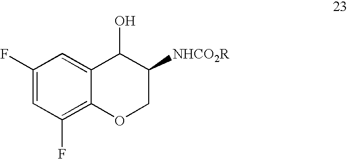
followed by the hydrogenolysis of the compound of formula 23 in a solvent comprising a C₁-C₆ alkylsulfonic acid, wherein R is alkyl or aryl, wherein: the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; and the term halogen means fluorine, chlorine, bromine or iodine.

2. A process for preparing a compound of formula 22′: 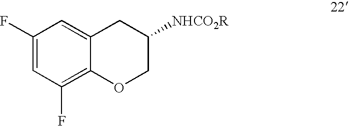
comprising reducing a compound of formula 21′: 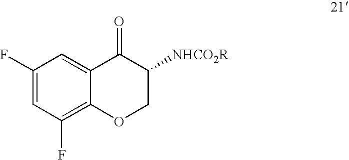
to produce a compound of formula 23′: 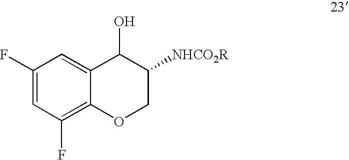
followed by the hydrogenolysis of the compound of formula 23′ in a solvent comprising a C₁-C₆ alkylsulfonic acid, wherein R is alkyl or aryl, wherein: the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; and the term halogen means fluorine, chlorine, bromine or iodine.

3. A process according to claim 1 or claim 2 wherein R is ethyl, methyl, tBu or benzyl.

4. A process according to claim 1, 2 or 3, wherein the C₁-C₆ alkylsulfonic acid is methane sulfonic acid.

5. A process according to any preceding claim, wherein the solvent used in the hydrogenolysis further includes a chlorinated solvent.

6. A process according to claim 5, wherein the ratio of the chlorinated solvent to the C₁ to C₆ alkyl sulfonic acid is not more than 10:1.

7. A process according to claim 5, wherein the ratio of the chlorinated solvent to the C₁ to C₆ alkyl sulfonic acid is about 3:1.

8. A process according to any preceding claim, wherein the reduction of the compound of formula 21 or 21′ is carried out using a borohydride compound in a suitable solvent.

9. A process according to claim 8, wherein the borohydride compound is sodium borohydride.

10. A process according to claim 8 or 9, wherein the solvent is a lower alcohol optionally in combination with THF.

11. A process according to claim 8 or 9, wherein the solvent is methanol optionally in combination with THF.

12. A process according to any preceding claim, wherein the hydrogenolysis step is carried out in the presence of a catalyst.

13. A process according to claim 12, wherein the catalyst is a palladium catalyst.

14. A process according to any preceding claim, wherein the hydrogenolysis step is carried out in a hydrogen atmosphere.

15. A process according to any preceding claim, wherein the solvent used in the hydrogenolysis comprises an alkyl halide in combination with methanesulfonic acid.

16. A process according to any preceding claim, wherein the solvent used in the hydrogenolysis comprises dichloromethane in combination with methanesulfonic acid.

17. A process for preparing a compound of formula 8: 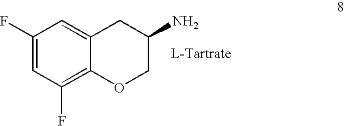
comprising subjecting a compound of formula 22 to alkaline hydrolysis:

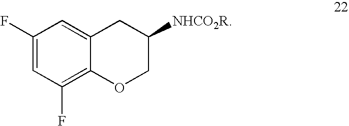

18. A process according to claim 17, wherein the compound of formula 22 is formed by a process according to any one of claims 1 or 3 to 16.

19. A process according to claim 17 or 18, wherein the alkaline hydrolysis step is carried out in the presence of an alkaline earth metal hydroxide or an alkali metal hydroxide; an alcohol having 1 to 6 carbon atoms; and L-tartaric acid.

20. A process for preparing a compound of formula 8′: 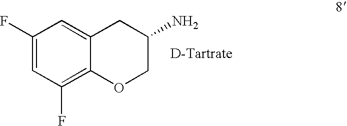
comprising subjecting a compound of formula 22′ to alkaline hydrolysis:

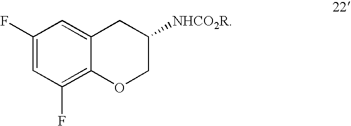

21. A process according to claim 20, wherein the compound of formula 22′ is formed by a process according to any one of claims 2 to 16.

22. A process according to claim 20 or 21, wherein the alkaline hydrolysis step is carried out in the presence of an alkaline earth metal hydroxide or an alkali metal hydroxide; an alcohol having 1 to 6 carbon atoms; and D-tartaric acid.

23. A process according to claim 19 or 22, wherein the alkali metal hydroxide is potassium hydroxide, and the alcohol is methanol.

24. A process for preparing a compound of formula 14: 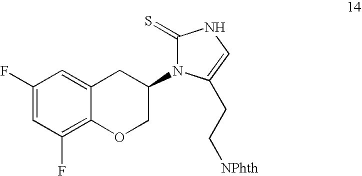
comprising forming a compound of formula 8 by a process according to any one of claim 17, 18, 19 or 23, then reacting the compound of formula 8 with a compound of formula 13:

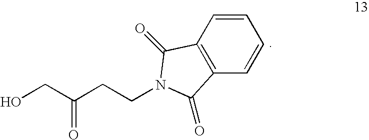

25. A process for preparing a compound of formula 14′: 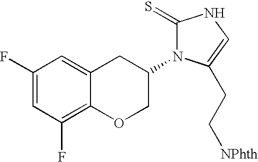
comprising forming a compound of formula 8′ by a process according to any one of claim 20, 21, 22 or 23, then reacting the compound of formula 8′ with a compound of formula 13:

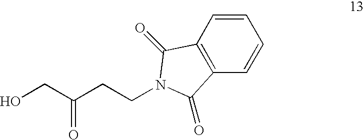

26. A process according to claim 24 or 25, wherein the reaction is carried out in the presence of an alkali metal isothiocyanate and an organic acid.

27. A process according to claim 26, wherein the alkali metal isothiocyanate is potassium isothiocyanate.

28. A process according to claim 26 or 27, wherein the organic acid is acetic acid.

29. A process for preparing a compound of formula 1: 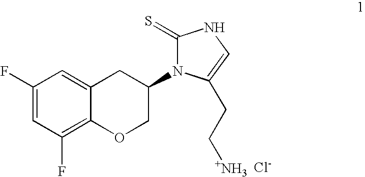
comprising preparing a compound of formula 14 by a process according to claim 24, 26, 27 or 28, then converting the compound of formula 14 to the compound of formula 1 with an alkali metal borohydride in the presence of a solvent, followed by adding HCl then recovering the compound of formula 1.

30. A process for preparing a compound of formula 1′: 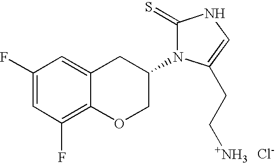
comprising preparing a compound of formula 14′ by a process according to claim 25, 26, 27 or 28, then converting the compound of formula 14′ to the compound of formula 1′ with an alkali metal borohydride in the presence of a solvent, followed by adding HCl then recovering the compound of formula U.

31. A process according to claim 29 or 30, wherein the alkali metal borohydride is sodium borohydride (NaBH₄).

32. A process according to claim 29, 30 or 31, wherein the solvent used in the conversion of compound 14 or 14′ is a mixture isopropyl alcohol, water and dichloromethane.

33. A process for preparing a compound of formula 22: 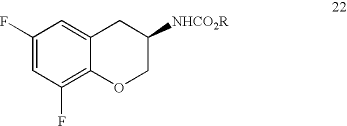
comprising hydrogenolysis of a compound of formula 21: 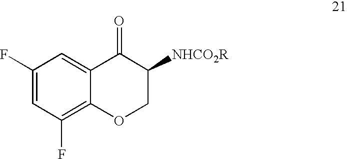
to produce the compound of formula 22, said hydrogenolysis being carried out in a solvent mixture comprising 3 volumes of a chlorinated solvent in combination with 1 volume of C₁-C₆ alkylsulfonic acid, preferably methanesulfonic acid, wherein R is alkyl or aryl, wherein: the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; and the term halogen means fluorine, chlorine, bromine or iodine.

34. A process for preparing a compound of formula 22′: 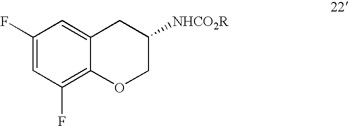
comprising hydrogenolysis of a compound of formula 21′: 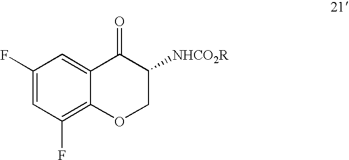
to produce the compound of formula 22′, said hydrogenolysis being carried out in a solvent mixture comprising 3 volumes of a chlorinated solvent in combination with 1 volume of C₁-C₆ alkylsulfonic acid, preferably methanesulfonic acid, wherein R is alkyl or aryl, wherein: the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; and the term halogen means fluorine, chlorine, bromine or iodine.

35. A process as claimed in claim 33 or claim 34 wherein R is ethyl, methyl, tBu or benzyl.

36. A compound of formula 4: 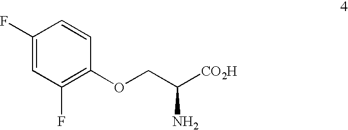
or a pharmaceutically acceptable salt thereof.

37. A compound of formula 4′: 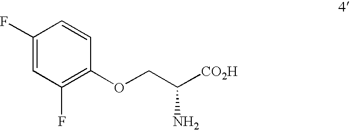
or a pharmaceutically acceptable salt thereof.

38. A compound of formula 20: 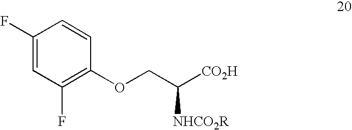
or a pharmaceutically acceptable salt thereof, wherein R is alkyl or aryl, wherein: the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; and the term halogen means fluorine, chlorine, bromine or iodine.

39. A compound of formula 20′: 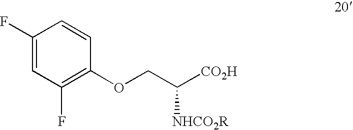
or a pharmaceutically acceptable salt thereof, wherein R is alkyl or aryl, wherein: the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; and the term halogen means fluorine, chlorine, bromine or iodine.

40. A compound of formula 21: 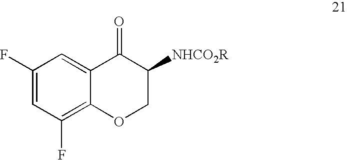
or a pharmaceutically acceptable salt thereof, wherein R is alkyl or aryl, wherein: the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; and the term halogen means fluorine, chlorine, bromine or iodine.

41. A compound of formula 21′: 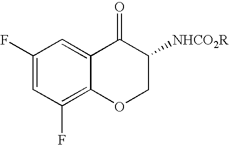
or a pharmaceutically acceptable salt thereof, wherein R is alkyl or aryl, wherein: the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; and the term halogen means fluorine, chlorine, bromine or iodine.

42. A compound of formula 23: 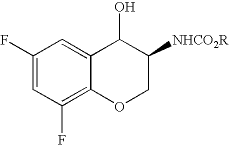
or a pharmaceutically acceptable salt thereof, wherein R is alkyl or aryl, wherein: the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; and the term halogen means fluorine, chlorine, bromine or iodine.

43. A compound of formula 23′: 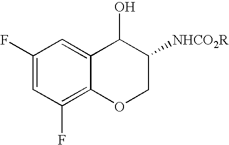
or a pharmaceutically acceptable salt thereof, wherein R is alkyl or aryl, wherein: the term alkyl means hydrocarbon chins, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; and the term halogen means fluorine, chlorine, bromine or iodine.

44. A compound according to any of claims 36 to 41, wherein R is ethyl, methyl, tBu or benzyl.

45. A compound of formula 8:

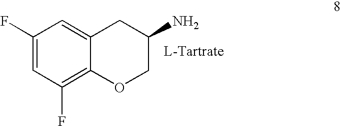

46. A compound of formula 8′: [chemical structure]
